(19) United States
(12) Patent Application Publication
(10) Pub. No.: US 2021/0267819 A1
(43) Pub. Date: Sep. 2, 2021

(54) CELLULOSE NONWOVEN LAMINATE HAVING 3D EMBOSSING

(71) Applicant: Kimberly-Clark Worldwide, Inc., Neenah, WI (US)

(72) Inventors: InYoung Sa, Seongnam-si (KR); SangMin Lee, Seoul (KR); SeoYeon Son, Seongnam-si (KR)

(21) Appl. No.: 17/256,459

(22) PCT Filed: Jun. 28, 2019
(86) PCT No.: PCT/KR2019/007850

§ 371 Date: Dec. 28, 2020

(30) Foreign Application Priority Data
Jun. 29, 2018 (KR) 10-2018-0075965

(51) Int. Cl. A61F 13/511; D04H 3/11; D04H 1/492; D04H 3/015

(52) U.S. Cl. CPC A61F 13/51108; D04H 3/11; D10B 2201/02; D04H 3/015; A61F 2013/51178; D04H 1/492

(57) ABSTRACT

The present disclosure presents a nonwoven laminate having two or more layers that have a 3D embossed hydrophobic cellulose-based fiber spunlace as a top layer. Also disclosed is the top layer having a z-directional topography where the base of the top layer is attached to a second carrier sheet layer(s) by adhesive, pressure bonding, thermal bonding, hole punching, or similar bonding.

### TECHNICAL FIELD

The current disclosure pertains to a three-dimensional (3D) nonwoven laminated absorbent article with two or more layers that comprise a hydrophobic cellulose-based spunlace top layer.

### BACKGROUND ART

Fibrous nonwoven web materials are in wide use in a number of applications including but not limited to absorbent structures and wiping products, many of which are disposable. In particular, such materials are commonly used in personal care absorbent articles such as diapers, diaper pants, training pants, feminine hygiene products, adult incontinence products, bandages and wiping products such as baby and adult wet wipes. They are also commonly used in cleaning products such as wet and dry disposable wipes which may be treated with cleaning and other compounds which are designed to be used by hand or in conjunction with cleaning devices such as mops. Yet a further application is with beauty aids such as cleansing and make-up removal pads and wipes.

In many of these applications, three-dimensionality and increased surface area are desirable attributes. This is particularly true with body contacting materials for the aforementioned personal care absorbent articles and cleaning products. One of the main functions of personal care absorbent articles is to absorb and retain body exudates such as blood, menses, urine and bowel movements.

More specifically, cellulose fiber nonwoven materials have a high potential use in absorbent structures and wiping products. However, there are general issues using cellulose fiber nonwoven materials. For instance, the cellulose fiber holding fluid inside a body side liner does not perform well in comparison with synthetic fiber in terms of dryness and stain size. Infeirity of dryness and stain masking is the main reason why cellulose fiber products have not been used in the market.

Additionally, due to the nature of cellulose fiber, it can only be produced by spunlacing process, which limits the aesthetics of the web highly dependent on the mechanical dimension of the pattern roll and waterjet process used during the spunlace process. As the pattern for spunlace is created by waterjet, the pattern that can be made through this process is very simple. Spunlaced cellulose fiber can be embossed and apertured as BCW webs, however, due to the thermal property of the fiber, the embossing does not maintain its 3D shape and easily becomes flat. Due to the limitation with pattern, any studies of cellulose fiber have been made with a plain, or a simple mesh type patterns for cotton product. As a summary, the problems that current cellulose-based spunlace material for absorbent body side liner for example have are:
Wet and larger stain due to the hydrophilicity of cellulose fiber, compared with synthetic nonwovens (PE/PP/PET)
Limitation on the differentiation against competitor product, due to the limited types/resolution of the pattern obtained by spunlacing process
Pattern often gets collapsed/less visible by the tension applied to the material, due to the high machine speed.

### DISCLOSURE OF INVENTION

### Technical Problem

As a result, there is a still a need for both a material and a process which provide three-dimensional characteristics that meet the aforementioned needs of a cellulose fiber nonwoven material.

### Solution to Problem

The current disclosure pertains to a three-dimensional (3D) nonwoven laminated absorbent article with two or more layers that comprise a hydrophobic cellulose-based spunlace top layer. The absorbent article also comprises a nonwoven carrier sheet layer wherein the body facing portion of the carrier sheet is attached to the non-body facing portion of the top layer; and optionally one or more nonwoven carrier sheet layers that are attached to the non-facing body portion of the carrier sheet. The process for making a 3D nonwoven laminated absorbent article is also disclosed.

In one embodiment of the present disclosure, a nonwoven laminated absorbent article with two or more layers is disclosed. Specifically, such an absorbent article comprises a hydrophobic cellulose-based spunlace top layer. The top layer comprises embossed raised regions with each region between about 0.1 mm to about 20 mm and more specifically from about 2 mm to about 8 mm in diameter and a height of at least about 0.5 mm. The absorbent article also comprises a nonwoven carrier sheet layer where the body facing portion of the carrier sheet is attached to the non-body facing portion of the top layer. The absorbent article optionally comprises one or more nonwoven carrier sheet layers that are attached to the non-facing body portion of the carrier sheet.

In another embodiment of the present disclosure, a process for making a nonwoven laminated absorbent article with two or more layers is disclosed. Specifically, the process comprises embossing a hydrophobic cellulose-based spunlace on to a top layer. The process also comprises attaching a body facing portion of a nonwoven carrier sheet layer to the non-body facing portion of the top layer. The process also optionally comprises attaching one or more body facing nonwoven carrier sheet layers to the non-facing body portion of the carrier sheet.

### BRIEF DESCRIPTION OF DRAWINGS

The disclosure may be more completely understood in consideration of the following detailed description of various embodiments in connection with the accompanying drawing, in which:

FIG. 1 shows the center of a raised region of the top of a cellulose fiber spunlaced layer collapse when the embossing is 10 mm width (red dash) versus when the embossing did not collapse for a 5 mm width.

FIG. 2 shows stain size comparison between semi-hydrophobic treated, and non-treated hydrophilic cotton spunlace.

FIG. 3 depicts a softness test result showing the plain cotton with 3D+× embossing as the softest in comparison to mesh patterned cotton.

FIG. 4 shows pattern images comparing conventional spunlace versus 3D spunlace laminate tensions.

FIG. 5 shows a schematic drawing of fluid discharge flows and fluid absorption of a 3D-embossed cellulose spunlace laminate.

FIG. 6 depicts where fluid tends to follow which is at the aperture line marked as the white dash. The aperture line shows where the highest wettability occurs.

FIG. 7 depicts a re-wet comparison between hydrophobic 2D mesh, hydrophilic 3D, and three samples of hydrophobic and 3D lamination shows significant improvement of rewet regardless of the pattern.

FIG. 8A shows stain size comparison of a 2D Hydrophobic Mesh, 3D Hydrophilic laminate and a 3D Hydrophobic 3D laminate.

FIG. 8B shows stain size image analysis results based on FIG. 8A.

### MODE FOR THE INVENTION

When introducing elements of the present disclosure or the preferred embodiment(s) thereof, the articles “a”, “an”, and “the” are intended to mean that there are one or more of the elements.

The terms “comprising”, “including” and “having” are intended to be inclusive and mean that there may be additional elements other than the listed elements.

The term “absorbent article” refers to devices that absorb and contain body exudates, and, more specifically, refers to devices that are placed against or in proximity to the body of the wearer to absorb and contain the various exudates discharged from the body. Absorbent articles may include pantiliners, sanitary napkins, interlabial devices, adult incontinence devices, bandages, wipes, diapers, training pants, undergarments, other feminine hygiene products, breast pads, care mats, bibs, wound dressing products, and the like.

The term “nonwoven” is a manufactured sheet, web or batt of directionally or randomly orientated fibers, bonded by friction, and/or cohesion and/or adhesion, excluding paper and products which are woven, knitted, tufted, stitch-bonded incorporating binding yarns or filaments, or felted by wet-milling, whether or not additionally needled. Nonwovens may include hydroentangled nonwovens. The fibers may be of natural or man-made origin and may be staple or continuous filaments or be formed in situ. Commercially available fibers have diameters ranging from less than about 0.001 mm to more than about 0.2 mm and they come in several different forms: short fibers (known as staple, or chopped), continuous single fibers (filaments or monofilaments), untwisted bundles of continuous filaments (tow), and twisted bundles of continuous filaments (yarn). Nonwoven fabrics can be formed by many processes such as meltblowing, spunbonding, solvent spinning, electrospinning, and carding. The basis weight of nonwoven fabrics is usually expressed in grams per square meter (gsm).

Absorbent articles typically include an outercover constructed from a laminate of a liquid impermeable film and a nonwoven fabric constructed from hydrophobic polymeric fibers.

As used herein, the term “spunlace” generally refers to a non-woven cloth, it is the direct use of polymer slices, short fibers or filaments into a network of fiber by air or mechanical, spunlace, acupuncture, or hot-rolled reinforcement, and finally after finishing the formation of spunlace nonwoven fabric.

In accordance with the present disclosure, it has been found, surprisingly and unexpectedly, that a 3D material structure of cellulouse-based spunlace and surge laminate may achieve superior dryness, cleanliness, and softness. The industrial norm is that cellulose spunlace is inferior to Through Air Bonded Carded Web (TABCW) nonwoven for dryness, softness and stain masking, however, the disclosure herein resulted in superior bench test result against a TABCW top sheet. TABCW combines two technologies: (1) carding and (2) thermal bonding.

Two important attributes of the disclosure herein are:

a nonwoven laminate having two or more layers that has a cellulose fiber based spunlace as a top layer (body facing), wherein such celluose fiber has less hydrophilicity than the second nonwoven carrier sheet layer and optionally any additional sheet layers that may be attached to the second carrier sheet layer; and

a top layer (body-facing) that has a z-directional topography, and the non facing body side of the top layer is attached to the second (carrier sheet) layer(s) by adhesive, pressure bonding, thermal bonding, hole punching, or similar bonding.

A film-web laminate may be formed from a nonwoven web overlying a spunlace layer and/or carrier sheet(s) (secondary sheets to the top sheet). In one embodiment, for instance, the nonwoven web is thermally laminated to the spunlace layer and/or the carrier sheet to form the film-web laminate. However, any suitable technique can be utilized to form the laminate. Suitable techniques for bonding a film to a nonwoven web are described in U.S. Pat. No. 5,843,057 to McCormack; U.S. Pat. No. 5,855,999 to McCormack; U.S. Pat. No. 6,002,064 to Kobylivker, et al.; U.S. Pat. No. 6,037,281 to Mathis, et al.; and WO 99/12734, which are incorporated herein in their entirety by reference thereto for all purposes.

The film layer of the laminate is typically formed from a material that is substantially impermeable to liquids. For example, the film layer may be formed from a thin plastic film or other flexible liquid-impermeable material. In one embodiment, the film layer is formed from a polyethylene film having a thickness of from about 0.001 millimeter to about 0.10 millimeter, about 0.01 millimeter to about 0.05 millimeters. For example, a stretch-thinned polypropylene film having a thickness of about 0.015 millimeter may be thermally laminated to the nonwoven web.

In addition, the film layer may be formed from a material that is impermeable to liquids, but permeable to gases and water vapor (i.e., “breathable”). This permits vapors to pass through the laminate, but still prevents liquid exudates from passing through the laminate. The use of a breathable laminate is especially advantageous when the laminate is used as an outercover of an absorbent article to permit vapors to escape from the absorbent core, but still prevents liquid exudates from passing through the outer cover. For example, the breathable film may be a microporous or monolithic film.

The film may be formed from a polyolefin polymer, such as linear, low-density polyethylene (LLDPE) or polypropylene. Examples of predominately linear polyolefin polymers include, without limitation, polymers produced from the following monomers: ethylene, propylene, 1-butene, 4-methyl-pentene, 1-hexene, 1-octene and higher olefins as well as copolymers and terpolymers of the foregoing. In addition, copolymers of ethylene and other olefins including butene, 4-methyl-pentene, hexene, heptene, octene, decene, etc., are also examples of predominately linear polyolefin polymers.

Material Requirements

Hydrophobic Cellulose-Based Nonwoven Fiber Top Layer

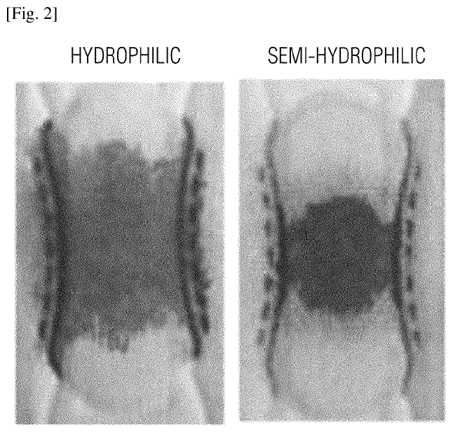

A top layer is composed of hydrophobic (to repel or mix with a liquid) cellulose-based fibers with a plain or staggered pattern. The hydrophobic cellulose-based fiber material contributes to a reduction of dryness and smaller stain size. Please see FIG. 2 depicting a hydrophilic versus semi-hydrophobic layer.

Generally, nonwoven cellulous fibers are fiber like materials made with ether or esters of cellulose, which may be obtained from the bark, wood or leaves of plants, or from a plant-based material. Besides cellulose, these fibers are compound of hemicellulose and lignin, and different percentages of these components are responsible for different mechanical properties observed. Cotton is one example of a nonwoven cellulose fiber used herein. Any nonwoven cellulose fiber may be applied herein.

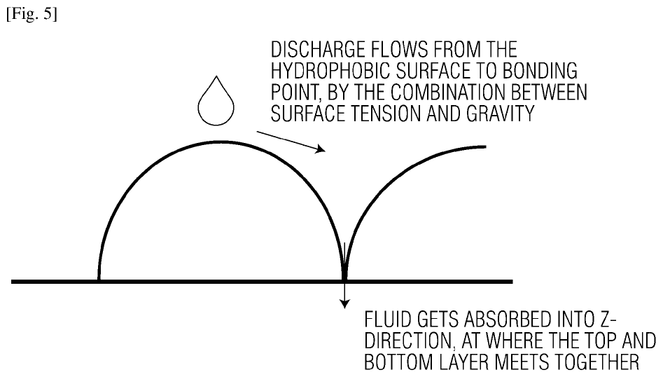

The top layer defined herein comprises of a nonwoven cellulose-based fiber may not be 100% hydrophobic. As disclosed in FIG. 5, a liquid flows from the hydrophobic surface in a z-direction, orthogonal to the x and y directions, at where the top and bottom layers meet. The liquid flows in a z-direction by a combination of surface tension and gravity. Accordingly, it is advantageous to have some porosity in the hydrophobic top layer.

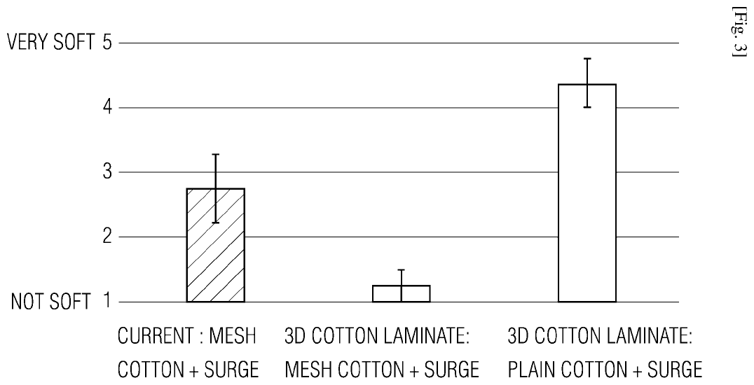

Surprisingly and unexpectedly, a 3D structure made of plain cellulose fiber spunlace sheet shows better softness as well as visual differentiation compared to patterned material. In FIG. 3, softness test result depicted indicate that a 3D structure leveraging plain cotton spunlace presents better softness than mesh patterned cotton.

Carrier Sheet Attached to the Top Layer

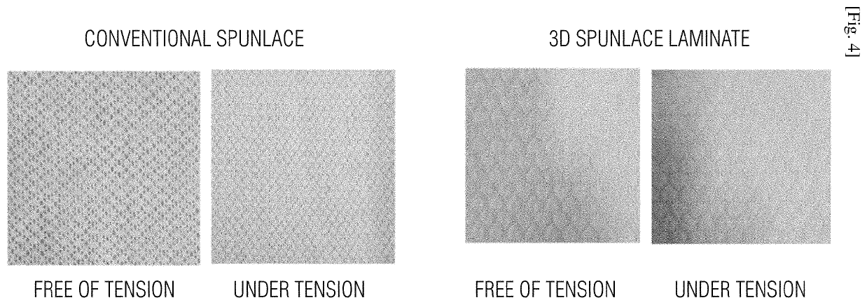

Any sheets with wettable property to body fluid, similar or higher mechanical strength than the top layer, preferably composes of high-denier fiber for ease of fluid transfer. FIG. 4 shows how tension impacts on the pattern of the conventional material versus the current disclosure. The carrier sheets (or second layer(s) sheets) are attached to the top layer by adhesive, bonding or by any other similar way. The attachment of the carrier sheet occurs at the non-body facing side of the top layer.

Structural Requirements

Top layer is spunlace and made of cellulouse fiber such as cotton. The spunlace is embossed upward to physically separate it from the bottom layer thus forming a 3D article. In order for the top layer 3D bump to be visible from the plain of the carrier sheet and be noticeable by touch, the height of the bump should be at least 0.5 mm. In order to provide ‘soft bulky’ feel, the cross-section of the staggered or plain embossing should have some curvature between the pillar and upper plain.

At about 10 mm in width is where the middle part of the top layer 3D bump begins to collapse due to gravity. By attaching a nonwoven carrier sheet on the bottom of the top layer thereby supporting the 3D pattern of the spunlace hydrophobic cellulose-based fiber, one may use less basis weight spunlace. Accordingly the amount of spunlace is determined by the width of the top layer. Optimally the amount of spunlace that may be used herein for the top layer is from about 15 gsm to about 50 gsm and from about 25 gsm to about 35 gsm. The amount of spunlace used on each of the carrier sheets is from about 10 gsm to about 50 gsm generally and from about 15 gsm to about 35 gsm more specifically. See. Table 1.

The bottom of the raised portion of the top layer (non-facing body side) and top of bottom layer (carrier sheet) are bonded together, by adhesive, pressure, temperature, or by aperturing to ensure that the base of the raised portions of the top layer are attached to the carrier sheet so that fluid acquisition moves in a Z-direction from the top layer down. Please see FIG. 5. The parameter ranges for pressure and temperature disclosed herein are ranges known in the art for bonding two or more nonwoven cellulose-based fiber layers together.

Size of the Embossed Cell

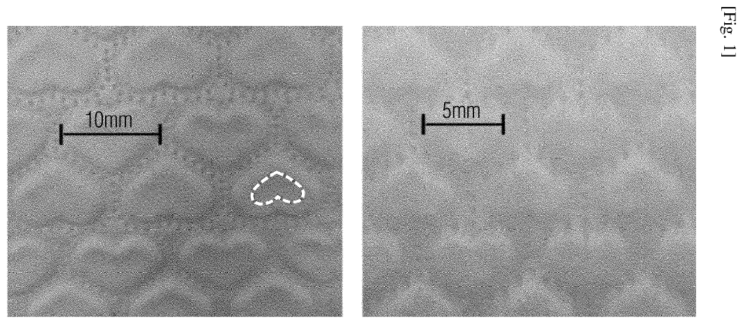

The optimal diameter width of a single embossed topology cell (spunlace cellulose fiber top layer) is from about 0.1 mm to about 20 mm, 0.5 mm to about 10 mm, about 1 mm to about 9 mm, 2 mm to about 8 mm to ensure the 3D embossed topology works for dryness. As the embossing topology layer reaches 10 mm in diameter in any direction the center collapses. See FIG. 1. As the topology top layer collapses, the top layer becomes closer to the carrier sheet layer, and thus a wetter surface is felt and seen.

Direction of the Aperture Lines

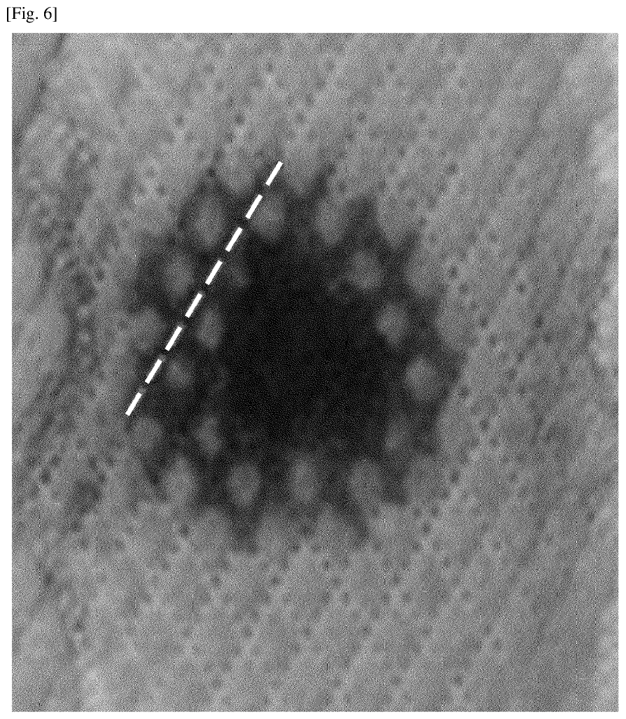

As the top layer is hydrophobic, fluid tends to follow the aperture line and is absorbed into the core absorption layer. Please see FIG. 6. In order to inhibit accidental run-offs to a pad edge(s) for instance, aperture lines aligned to diagonal direction is more preferred than having lines parallel to Machine Direction/Cross Direction.

3D Embossing

3D embossing is applied only to the top layer, and the shape of the top layer is supported by the carrier sheet underneath by adhesive lamination, for example, in order to physically separate the embossed cells from the bottom carrier layer. There are three different embossing/lamination methodologies that are presented in table 1.

TABLE 1

| | Example 1 | Example 2 | Example 3 |
|---|---|---|---|
| Top layer | 30 gsm Cotton | 30 gsm Cotton | 30 gsm Cotton |
| Bottom layer | 25 gsm TABCW | 20 gsm Spunbond | 30 gsm Cotton |
| Lamination | Adhesive | Spunlace | Embossing + |
| method | lamination | | ultrasonic |
| | | | bonding |

Test Results

A penetration rate test was performed using “Z-Date”. Z-Date is a synthetic menstrual fluid formulation available from PPG Industries, Inc. of Pittsburgh, Pa. that contains, on a weight percent basis, approximately 82.5 percent water, 15.8 percent polyvinyl pyrrolidone and 1.7 percent salts, coloring agents and surfactants. Z-Date has a viscosity of 17 centipoise and a surface tension of 53.5 dynes per centimeter. By using this Z-Date solution it was found that a 3D cellulose fiber cotton with material and structure requirements described herein above showed enhanced absorption characteristics.

FIG. 4 depicts pattern images comparing conventional spunlace versus 3D spunlace laminate tensions. Unlike the conventional spunlace, the 3D spunlace laminate images depict a laminate that is similar regardless if free of tension or under tension.

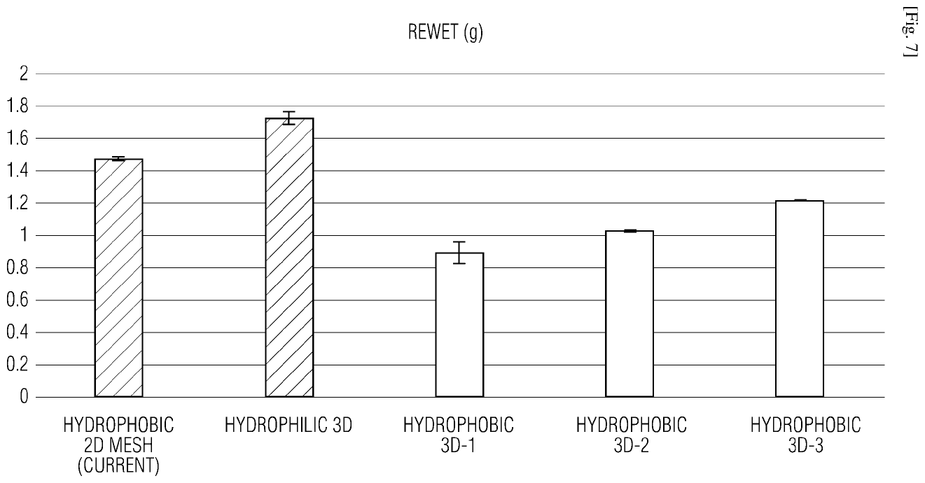

FIG. 7 indicates that all codes with 3D lamination and the hydrophobic treatment together (3 codes on the right) showed improved Rewet (dryness) regardless of the pattern.

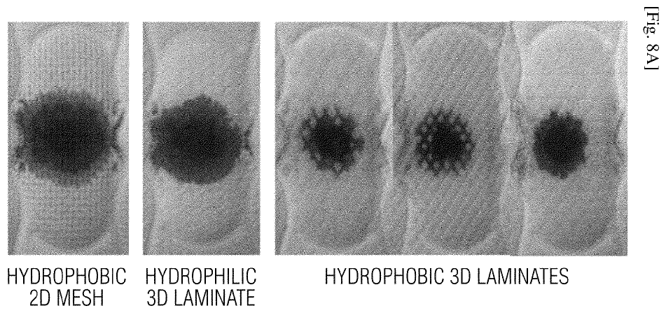

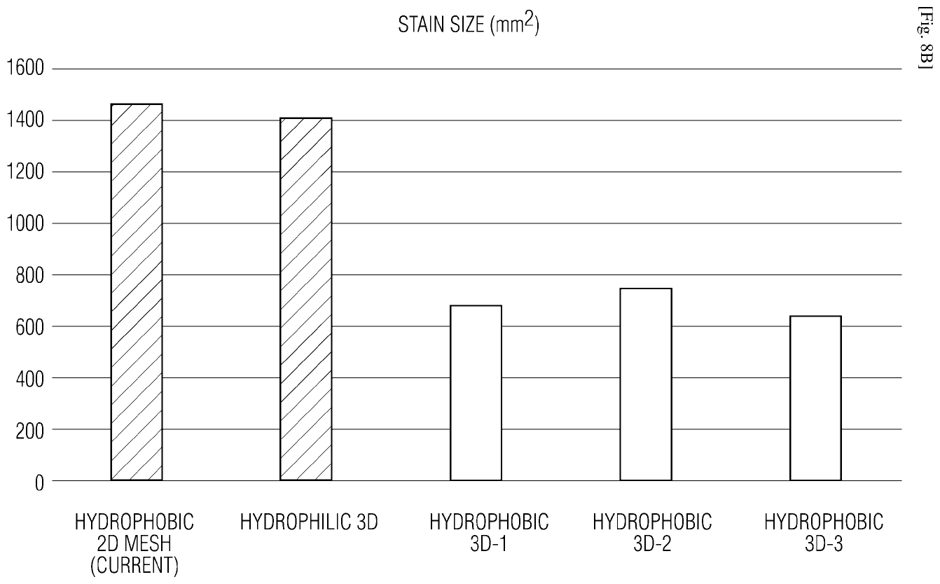

Combination of hydrophobic treatment and 3D lamination showed improvement on stain size as well, which is known perception of cleanliness by consumers. Please see FIGS. 8A and 8B. FIG. 8A shows stain size comparison of a 2D Hydrophobic Mesh, 3D Hydrophilic laminate and a 3D Hydrophobic 3D laminate. FIG. 8B shows a stain size image analysis results based on FIG. 8A.

The specification and example above are presented to aid in the complete and nonlimiting understanding of the invention disclosed herein. Since many variations and embodiments of the invention may be made without departing from its spirit and scope, the invention resides in the claims hereinafter appended.

### EMBODIMENTS

In a first embodiment of the present disclosure, a nonwoven laminated absorbent article with two or more layers is disclosed. Specifically, such an absorbent article comprises a hydrophobic cellulose-based spunlace top layer wherein the layer comprises embossed raised regions with each region between about 0.1 mm to about 20 mm in diameter and a height of at least about 0.5 mm; a nonwoven carrier sheet layer wherein the body facing portion of the carrier sheet is attached to the non-body facing portion of the top layer; and optionally one or more nonwoven carrier sheet layers that are attached to the non-facing body portion of the carrier sheet.

In an embodiment according to the preceding embodiment, wherein hydrophobic cellulose-based spunlace top layer is from about 2 mm to about 8 mm in diameter.

In an embodiment according to the preceding embodiment, wherein hydrophobic cellulose-based spunlace top layer is cotton.

In an embodiment according to the preceding embodiments, wherein the hydrophobic cellulose-based spunlace top layer and carrier sheet(s) are laminated by adhesive lamination, spunlace, or embossed and ultrasonic bonding.

In an embodiment according to the preceding embodiments, wherein the hydrophobic cellulose-based spunlace is plain.

In an embodiment according to the preceding embodiments, wherein the raised regions are in a staggered or plain pattern.

In an embodiment according to the preceding embodiments, wherein the hydrophobic cellulose-based spunlace top layer has less hydrophilicity than the attached nonwoven carrier sheet(s).

In an embodiment according to the preceding embodiments, wherein the top layer has a z-directional topography.

In an embodiment according to the preceding embodiments, wherein the top layer is attached to the carrier sheet layer by adhesive, pressure bonding, thermal bonding, hole punching or a similar bonding.

In an embodiment according to the preceding embodiments, wherein the carrier sheet layer is attached to the optional carrier sheet layer(s) by adhesive, pressure bonding, thermal bonding, hole punching or a similar bonding.

In an embodiment according to the preceding embodiments, wherein the absorbent article may be pantiliners, sanitary napkins, interlabial devices, adult incontinence devices, bandages, wipes, diapers, training pants, undergarments, other feminine hygiene products, breast pads, care mats, bibs, wound dressing products, and the like.

In an embodiment according to the preceding embodiments, wherein the top layer comprises about 25 gsm to about 35 gsm spunlace and the carrier sheets comprise from about 15 gsm to about 35 gsm.

In a second embodiment of the present disclosure, a process for making a nonwoven laminated absorbent article with two or more layers is disclosed. Specifically, the process comprises: embossing a hydrophobic cellulose-based spunlace on to a top layer; attaching a body facing portion of a nonwoven carrier sheet layer to the non-body facing portion of the top layer; and optionally attaching one or more body facing nonwoven carrier sheet layers to the non-facing body portion of the carrier sheet.

In an embodiment according to the preceding process embodiment, wherein embossing the hydrophobic cellulose spunlace top layer raised each region between about 0.1 mm to about 20 mm in diameter and a height of at least about 0.5 mm.

In an embodiment according to the preceding process embodiments, wherein hydrophobic cellulose-based spunlace top layer is cotton.

In an embodiment according to the preceding process embodiments, wherein the hydrophobic cellulose-based spunlace top layer and carrier sheet(s) are laminated by adhesive lamination, spunlace, or embossed and ultrasonic bonded.

In an embodiment according to the preceding process embodiments, wherein the hydrophobic cellulose-based spunlace is plain.

In an embodiment according to the preceding process embodiments, wherein the raised regions are in a staggered or plain pattern.

In an embodiment according to the preceding process embodiments, wherein the top layer has a z-directional topography.

In an embodiment according to the preceding process embodiments, wherein carrier sheet layer is attached to the optional carrier sheet layer(s) by adhesive, pressure bonding, thermal bonding, hole punching or a similar bonding.

In an embodiment according to the preceding process embodiments, wherein the absorbent article may be pantiliners, sanitary napkins, interlabial devices, adult incontinence devices, bandages, wipes, diapers, training pants, undergarments, other feminine hygiene products, breast pads, care mats, bibs, wound dressing products, and the like.

1. A nonwoven laminated absorbent article with two or more layers comprising:
a hydrophobic cellulose-based spunlace top layer wherein the layer comprises embossed raised regions with each region between about 0.1 mm to about 20 mm in diameter and a height of at least about 0.5 mm;
a nonwoven carrier sheet layer wherein the body facing portion of the carrier sheet is attached to the non-body facing portion of the top layer; and;
optionally one or more nonwoven carrier sheet layers that are attached to the non-facing body portion of the carrier sheet.

2. The nonwoven laminated absorbent article according to claim 1, wherein the hydrophobic cellulose-based spunlace top layer is from about 2 mm to about 8 mm in diameter.

3. The nonwoven laminated absorbent article according to claim 1, wherein hydrophobic cellulose-based spunlace top layer is cotton.

4. The nonwoven laminated absorbent article according to claim 1, wherein the hydrophobic cellulose-based spunlace top layer and carrier sheet(s) are laminated by adhesive lamination, spunlace, or embossed and ultrasonic bonding.

5. The nonwoven laminated absorbent article according to claim 1, wherein the hydrophobic cellulose-based spunlace is plain.

6. The nonwoven laminated absorbent article according to claim 1, wherein the raised regions are in a staggered or plain pattern.

7. The nonwoven laminated absorbent article according to claim 1, wherein the hydrophobic cellulose-based spunlace top layer has less hydrophilicity than the attached nonwoven carrier sheets.

8. The nonwoven laminated absorbent article according to claim 1, wherein the top layer has a z-directional topography.

9. The nonwoven laminated absorbent article according to claim 1, wherein the top layer is attached to the carrier sheet layer by adhesive, pressure bonding, thermal bonding, hole punching or a similar bonding.

10. The nonwoven laminated absorbent article according to claim 1, wherein the carrier sheet layer is attached to the optional carrier sheet layer(s) by adhesive, pressure bonding, thermal bonding, hole punching or a similar bonding.

11. The nonwoven laminated absorbent article according to claim 1, wherein the absorbent article may be pantiliners, sanitary napkins, interlabial devices, adult incontinence devices, bandages, wipes, diapers, training pants, undergarments, other feminine hygiene products, breast pads, care mats, bibs, wound dressing products, and the like.

12. The nonwoven laminated absorbent article according to claim 1, wherein the top layer comprises about 25 gsm to about 35 gsm spunlace and the carrier sheets comprise from about 15 gsm to about 35 gsm.

13. A process for making a nonwoven laminated absorbent article with two or more layers comprising:
embossing a hydrophobic cellulose-based spunlace on to a top layer;
attaching a body facing portion of a nonwoven carrier sheet layer to the non-body facing portion of the top layer; and;
optionally attaching one or more body facing nonwoven carrier sheet layers to the non-facing body portion of the carrier sheet.

14. The process for making a nonwoven laminated absorbent article with two or more layers according to claim 13, wherein embossing the hydrophobic cellulose spunlace top layer raised each region between about 0.1 mm to about 20 mm in diameter and a height of at least about 0.5 mm.

15. The process for making a nonwoven laminated absorbent article with two or more layers according to claim 13, wherein hydrophobic cellulose-based spunlace top layer is cotton.

16. The process for making a nonwoven laminated absorbent article with two or more layers according to claim 13, wherein the hydrophobic cellulose-based spunlace top layer and carrier sheet(s) are laminated by adhesive lamination, spunlace, or embossed and ultrasonic bonded.

17. The process for making a nonwoven laminated absorbent article with two or more layers according to claim 13, wherein the hydrophobic cellulose-based spunlace is plain.

18. The process for making a nonwoven laminated absorbent article with two or more layers according to claim 13, wherein the raised regions are in a staggered or plain pattern.

19. The process for making a nonwoven laminated absorbent article with two or more layers according to claim 13, wherein the top layer has a z-directional topography.

20. The process for making a nonwoven laminated absorbent article with two or more layers according to claim 13, wherein carrier sheet layer is attached to the optional carrier sheet layer(s) by adhesive, pressure bonding, thermal bonding, hole punching or a similar bonding.

21. The process for making a nonwoven laminated absorbent article with two or more layers according to claim 13, wherein the absorbent article may be pantiliners, sanitary napkins, interlabial devices, adult incontinence devices, bandages, wipes, diapers, training pants, undergarments, other feminine hygiene products, breast pads, care mats, bibs, wound dressing products, and the like.
